(19) United States
(12) Patent Application Publication
(10) Pub. No.: US 2010/0216877 A1
(43) Pub. Date: Aug. 26, 2010

(54) Storage Stable Prostaglandin Product

(75) Inventors: Rajesh Kshirsagar, Bangalore (IN); Chandrashekar Kadam, Bangalore (IN); Sachin Jain, Bangalore (IN); SM Mudda, Bangalore (IN)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: MICRO LABS LIMITED, Bangalore (IN)

(21) Appl. No.: 12/707,744

(22) Filed: Feb. 18, 2010

(30) Foreign Application Priority Data
Feb. 20, 2009 (IN) 377/CHE/2009

(51) Int. Cl. A61K 31/557; A61K 31/5575; A61P 27/06; B65D 1/00

(52) U.S. Cl. 514/530; 514/573; 514/622; 428/36.92

(57) ABSTRACT

A prostaglandin composition comprising prostaglandin and a low-density polyethylene container are disclosed. The prostaglandin compositions are stable in polyethylene containers over longer period of time.

### PRIORITY

This application claims the benefit of Indian Patent Application No. 377/CHE/2009 entitled “STORAGE STABLE PROSTAGLANDIN PRODUCT”, filed Feb. 20, 2009, which application is hereby incorporated by reference in its entirety.

### TECHNICAL FIELD OF THE INVENTION

The present invention provides a container and a stable method for storing a pharmaceutical composition comprising prostaglandin(s) wherein the method has the step of storing the prostaglandin composition in a polyethylene container, preferably low density polyethylene (LDPE), still preferably LDPE container having Purell PE 3020 D resin by using Blow Fill Seal (BFS) technology.

### BACKGROUND OF THE INVENTION

Glaucoma, an eye disorder afflicting various mammals, including primates, is characterized by increased intraocular pressure (ocular hypertension). In humans, such ocular hypertension is caused by an imbalance between the rate of secretion of aqueous humor by the ciliary epithelium into the anterior and posterior chambers of the eyes and the rate of outflow or drainage of the aqueous humor from the anterior and posterior chambers, primarily via the canal of Schlemm. It is generally believed that obstruction of the aqueous humor drainage is the primary cause of the imbalance.

Chronic glaucoma typically results in slow, progressive loss of visual fields, and, if not controlled, ultimately convert in blindness. Different active compounds are available to treat glaucoma, including various prostaglandins.

Prostaglandins have low water solubility, and are generally unstable. Attempts have been made to solubilize and stabilize various prostaglandins by complexing them with different cyclodextrins. See, for example: EP 330 511 A2 (Ueno et al.) and EP 435 682 A2 (Wheeler). These attempts have met with varying success.

Containers for ophthalmic products serve several purposes; facilitate manufacturing; maintain product protection, including sterility and freedom from Pyrogen; allow inspection of contents; permit shipping and storage; and provide convenient use. The container components for ophthalmic products must be considered as integral part of products because they can dramatically affect product stability, potency, toxicity and safety, and therefore must be evaluated carefully with variety of tests before selecting for particular active containing composition.

The widely used container components for ophthalmic product are glass and plastic however the use of glass containers has diminished and use of plastic containers have been favored because they weigh less, are more resistant to shock and other mechanical influences, cost less, and offer more design possibilities. Polyethylene preferably LDPE, that is, low-density polyethylene without or with additives, and polypropylene are the plastics required by the European Pharmacopoeia.

Polypropylene is known to be stronger, stiffer, and more high temperature-resistant than low-density polyethylene. However, polypropylene has a poorer resistance to oxidation agents such as oxygen and acids, which can lead to fissures and yellowing of the plastic. Also polypropylene does not provide superior flexibility and proccessability as compared to polyethylene and hence it is not a first choice as containers for sterile compositions, especially for blow fill seal technology. Also polypropylene is not a cost effective option as compared to polyethylene.

U.S. Pat. No. 6,235,781 (Weiner) ['781] discloses pharmaceutical products containing an aqueous prostaglandin composition packaged in polypropylene containers. According to '781 aqueous prostaglandin compositions packaged in polypropylene containers are more stable than those packaged in polyethylene containers. '781 further teaches that the stability of the prostaglandin formulations is affected by polyethylene containers (LDPE) as compared to polypropylene containers at different stability conditions.

Further PCT application WO 2002/022106 (Wong) discloses that unless refrigerated (2-8° C.), lipid soluble prostaglandin derivatives and analogues show unacceptable stability in standard low-density polyethylene (LDPE) containers. The requirement that the ophthalmic preparation be refrigerated greatly reduces the availability of the treatment to those in less developed parts of the world. Furthermore, even where available, refrigeration of the preparation increases the cost of the treatment to the patient, and thus, further reduces its availability to those in need.

There, therefore, exists a need in the art for a method for storing prostaglandin preparation using cost effective container components over longer periods of time

### SUMMARY OF THE INVENTION

The invention therefore provides a container and a method of increasing the stability of a pharmaceutical composition comprising an active compound selected from a prostaglandin, a prostaglandin derivative, or a prostaglandin analogue wherein the method has the step of providing the pharmaceutical composition, especially an ophthalmic composition, in a container produced from polyethylene, preferably LDPE, still preferably LDPE container having Purell PE 3020 D resin.

The invention, in addition, provides a container and a method of increasing the stability of an ophthalmic composition comprising travoprost, latanoprost, bimatoprost, tafluprost, wherein the container is made from polyethylene, preferably LDPE, still preferably LDPE container having Purell PE 3020 D resin prepared using BFS technology.

### DETAILED DESCRIPTION OF THE INVENTION

The prostaglandins, which may be utilized in the present invention, include all pharmaceutically acceptable prostaglandins, their derivatives and analogues, and their pharmaceutically acceptable esters and salts. Such prostaglandins include the natural compounds: PGE₁, PGE₂, PGE₃, PGFα, PGF₂α, PGF₃α, PGD₂ and PGI₂ (prostacyclin), as well as analogues and derivatives of these compounds which have similar biological activities of either greater or lesser potencies. Analogues of the natural prostaglandins include but are not limited to: alkyl substitutions (e.g., 15-methyl or 16,16-dimethyl), which confer enhanced or sustained potency by reducing biological metabolism or alter selectivity of action; saturation (e.g., 13,14-dihydro) or unsaturation (e.g., 2,3-didehydro, 13,14-didehydro), which confer sustained potency by reducing biological metabolism or alter selectivity of action; deletions or replacements (e.g., 11-deoxy, 9-deoxo-9-methylene), chloro (or halogen) for oxygen (e.g., 9.beta.-chloro), oxygen for carbon (e.g., 3-oxa), lower alkyl for oxygen (e.g., 9-methyl), hydrogen for oxygen (e.g., 1-CH.sub.2OH,1-CH.sub.2OAcyl) which enhance chemical stability and/or selectivity of action; and .omega.-chain modifications (e.g., 18,19,20-trinor-17-phenyl, 17,18,19,20-tetranor-16-phenoxy), which enhance selectivity of action and reduce biological metabolism. Derivatives of these prostaglandins include all pharmaceutically acceptable salts and esters, which may be attached to the 1-carboxyl group or any of the hydroxyl groups of the prostaglandin by use of the corresponding alcohol or organic acid reagent, as appropriate. It should be understood that the terms “analogues” and “derivatives” include compounds that exhibit functional and physical responses similar to those of prostaglandins per se. The prostaglandins suitable for use in the compositions of the present invention can be selected from group consisting of travoprost, latanoprost, bimatoprost, tafluprost and the like.

The present inventors have surprisingly found against the teachings of the prior art i.e. the prostaglandins are not stable in the polyethylene containers.

The present inventors have now found that a composition comprising a prostaglandin can be made stable in polyethylene container by using suitable grade of polyethylene resin for container system.

The present inventors have further found that addition of suitable additives to polyethylene resin used to prepare container which are compatible with active further contributes in increasing stability.

The present inventors have further found that dose of gamma sterilization used for sterilization of polyethylene container also has impact on stability of prostaglandin product packaged in polyethylene container.

The present inventors have further found that gamma sterilization of 15-25 kGy for low density polyethylene is optimum for maintaining and increasing stability of prostaglandin packaged in polyethylene container. The gamma sterilization beyond this limit tends to increase adsorption and hence fall in assay or potency of the prostaglandin product.

The present inventors further found that the stability of prostaglandin compositions preferably travoprost, latanoprost, bimatoprost, tafluprost compositions can be increased when these compositions were packaged in LDPE containers; preferably LDPE containers prepared from Purell PE 3020 D resins preferably using BFS technology.

The stability of prostaglandins compositions was further increased when the sterilization was done using gamma radiation of 15-25 kGy or without using gamma radiation. Thus the gamma radiation was found to have impact on stability of preferably prostaglandin compositions, still preferably Travoprost compositions.

The present inventors further found that preservative adsorption or loss in prostaglandin composition can be prevented to significant level by packaging prostaglandin compositions in polyethylene container, preferably in LDPE containers, still preferably in Purell PE 3020 D container and preferably with gamma sterilization of 15-25 KGy.

Thus, the ophthalmic composition of the present invention has preferably travoprost in a container prepared from LDPE container having Purell PE 3020 D resin produced using blow fill seal (BFS) technology and having sufficient squeeze-ability to dispense drops by digital manipulation of the bottle by the user.

In one embodiment, the present invention provides a method of increasing the stability of a pharmaceutical composition comprising an active compound selected from a prostaglandin, a prostaglandin derivative, or a prostaglandin analogue wherein the method has the step of providing the pharmaceutical composition in a container produced from polyethylene.

In another embodiment, the present invention provides a method of increasing the stability of a pharmaceutical composition comprising an active compound selected from a prostaglandin, a prostaglandin derivative, or a prostaglandin analogue, and preservative and pharmaceutically acceptable excipient wherein the method consists of the step of providing the pharmaceutical composition in a multi-dose container produced from polyethylene wherein the prostaglandin product is stable at room temperature up to 25° C. for more than twelve months.

In yet another embodiment, the present invention provides a method of increasing the stability of a pharmaceutical composition comprising an active compound selected from a prostaglandin, a prostaglandin derivative, or a prostaglandin analogue, and preservative and pharmaceutically acceptable excipients wherein the method consists of the step of providing the pharmaceutical composition in a multi-dose container produced from polyethylene wherein the prostaglandin product is stable without refrigeration at 2-8° C.

In yet another embodiment, the present invention provides a method of increasing the stability of prostaglandin composition comprising an active compound selected from a prostaglandin, a prostaglandin derivative, or a prostaglandin analogue, and preservative and pharmaceutically acceptable excipients wherein the method consists of the step of providing ophthalmic composition in multi-dose container produced by using BFS technology from LDPE container having Purell PE 3020 D resin.

In yet another embodiment, the present invention provides a method of increasing the stability of prostaglandin composition comprising an active compound selected from travoprost, latanoprost, bimatoprost, tafluprost and preservative and pharmaceutically acceptable excipients wherein the method consists of the step of providing the pharmaceutical composition, in a multi-dose container produced from polyethylene, preferably LDPE, still preferably LDPE having Purell PE 3020 D resin.

In yet another embodiment, the present invention provides a method of increasing the stability of prostaglandin composition comprising an active compound selected from travoprost, latanoprost, bimatoprost, tafluprost, and preservative and pharmaceutically acceptable excipients wherein the method consists of the step of providing the pharmaceutical composition, in a multi-dose container produced from polyethylene, preferably LDPE, still preferably LDPE container having Purell PE 3020 D resin wherein the composition is stable at 60° C. and 40° C./RH not more than 25% for more than six months or one year.

In yet another embodiment, the present invention provides a method of increasing the stability of prostaglandin composition comprising an active compound selected from travoprost, latanoprost, bimatoprost, tafluprost and preservative and pharmaceutically acceptable excipients wherein the method consists of the step of providing the pharmaceutical composition, in a multi-dose container produced from polyethylene, preferably LDPE, still preferably LDPE container having Purell PE 3020 D resin wherein the composition is stable at room temperature up to 25° C. for more than twelve months.

In yet another embodiment, the present invention provides a method of increasing the stability of prostaglandin composition comprising an active compound selected from travoprost, latanoprost, bimatoprost, tafluprost and preservative and pharmaceutically acceptable excipients wherein the method consists of the step of providing the pharmaceutical composition, in a multi-dose container produced from polyethylene, preferably LDPE, still preferably LDPE container having Purell PE 3020 D resin wherein the composition is stable without refrigeration at 2-8° C.

In yet another embodiment, the present invention provides a method of increasing the stability of prostaglandin composition comprising an active compound selected from travoprost, latanoprost, bimatoprost, tafluprost and preservative and pharmaceutically acceptable excipients wherein the method consists of the step of providing the pharmaceutical composition, in a multi-dose container produced from polyethylene, preferably LDPE, still preferably LDPE container having Purell PE 3020 D resin with gamma sterilization of 15-25 kGy.

In yet another embodiment, the present invention provides a method of increasing the stability of prostaglandin composition comprising an active compound selected from travoprost, latanoprost, bimatoprost, tafluprost and benzalkonium chloride and polylethoxylated castor oil wherein the method consists of the step of providing the pharmaceutical composition, in a container produced from LDPE having Purell PE 3020 D resin with gamma sterilization of 15-25 kGy.

In yet another embodiment, the present invention provides a method of increasing the stability of prostaglandin composition comprising an active compound selected from travoprost, latanoprost, bimatoprost, tafluprost and polylethoxylated castor oil wherein the method consists of the step of providing the pharmaceutical composition, in a container produced from polyethylene, preferably LDPE, still preferably LDPE container having Purell PE 3020 D resin wherein container is non-gamma sterilized.

In yet another embodiment, the present invention provides a method of increasing the stability of prostaglandin composition comprising travoprost, benzalkonium chloride and polylethoxylated castor oil wherein the method consist of the step of providing the composition, in a multi-dose container produced by BFS technology using Purell PE 3020 D resin.

In yet another embodiment, the present invention provides a method of increasing the stability of prostaglandin composition comprising bimatoprost, benzalkonium chloride and polylethoxylated castor oil wherein the method consists of the step of providing the bimatoprost composition in a multi-dose container produced by BFS technology using Purell PE 3020 D resin.

In yet another embodiment, the present invention provides a method of increasing the stability of prostaglandin composition comprising latanoprost, benzalkonium chloride and polylethoxylated castor oil wherein the method consists of the step of providing the latanoprost composition in a multi-dose container produced by BFS technology using Purell PE 3020 D resin.

In yet another embodiment, the present invention provides a method of increasing the stability of prostaglandin composition comprising tafluprost, benzalkonium chloride and polylethoxylated castor oil wherein the method consists of the step of providing the latanoprost composition in a multi-dose container produced by BFS technology using Purell PE 3020 D resin.

In yet another embodiment, the present invention provides a method of increasing the stability of prostaglandin composition comprising a prostaglandin, preservative and pharmaceutically acceptable excipient wherein the method comprises: packaging the prostaglandin composition in low-density polyethylene multi-dose container.

In yet another embodiment, the present invention provides a method of increasing the stability of an aqueous ophthalmic composition comprising a travoprost, preservative and pharmaceutically acceptable excipients wherein the method comprises: packaging the travoprost composition in low density polyethylene multi-dose container prepared using blow fill seal technology wherein the low density polyethylene resin is Purell PE 3020 D.

In yet another embodiment, the present invention provides method of increasing the stability of an aqueous ophthalmic composition comprising a latanoprost, preservative and pharmaceutically acceptable excipients wherein the method comprises: packaging the latanoprost composition in low density polyethylene multi-dose container prepared using blow fill seal technology wherein the low density polyethylene resin is Purell PE 3020 D.

In yet another embodiment, the present invention provides method of increasing the stability of an aqueous ophthalmic composition comprising a bimatoprost, preservative and pharmaceutically acceptable excipients wherein the method comprises: packaging the bimatoprost composition in low density polyethylene multi-dose container prepared using blow fill seal technology wherein the low density polyethylene resin is Purell PE 3020 D.

In yet another embodiment, the present invention provides method of increasing the stability of an aqueous ophthalmic composition comprising a tafluprost, preservative and pharmaceutically acceptable excipients wherein the method comprises: packaging the tafluprost composition in low density polyethylene multi-dose container prepared using blow fill seal technology wherein the low density polyethylene resin is Purell PE 3020 D.

The invention, in addition, provides a container for increasing the stability of prostaglandin composition comprising prostaglandin wherein the container is made of polyethylene, preferably LDPE, still preferably LDPE container having Purell PE 3020 D resin. The bottle does not substantially adsorb the active compound or preservative even when the composition is not refrigerated over a period between one and 18 months. The term “substantially” as used herein indicates less than 5 wt %, preferably less than 3 wt %.

Examples of suitable preservatives for multi-dose topically administrable ophthalmic formulations include: benzalkonium chloride, thimerosal, chlorobutanol, methyl paraben, propyl paraben, phenylethyl alcohol, edetate disodium, sorbic acid, Polyquad®. and other agents equally well known to those skilled in the art. Such preservatives, if present, will typically be employed in an amount between about 0.001 and about 1.0 wt. %.

The prostaglandin compositions packaged in polyethyelene containers according to the present invention can be adapted for any route of administration. Compositions adapted for topical administration to the ears, nose or eyes are preferred, with compositions prepared for topical administration to the eye being most preferred.

The pharmaceutically acceptable excipients according to present invention are formulatory ingredients, such as vehicles, surfactants, tonicity agents, and buffers. Many such formulatory ingredients are known.

As used herein “LDPE” means low density polyethylene. The preferred compositions are preferably packaged in the containers preferably produced by BFS technology or three piece container using LDPE resins selected from group consisting of Purell PE 1810 E, Purell PE 1840 H, Purell PE 3020 D, Purell PE 3040 D, Purell PE 3220 D, most preferably Purell PE 3020 D.

The preferred compositions are preferably packaged in the multi-dose containers produced by BFS technology. In BFS process the plastic is heated to semi-molten state and pushed through the parison assembly via a screw and temperatures controlled cylinder. The plastic is channeled through dies that may be multiple with one for each bottle or single oval or round, from which smaller vials will be formed. Air or nitrogen, sterile where necessary, flows through the assembly at all times to keep the plastic from collapsing on to extrude the resin are sporicidal.

The preferred compositions are preferably packaged in a “small volume” bottle. As used herein, the term “small volume” bottle shall mean a bottle of a size sufficient to hold a quantity of liquid medicine sufficient for 1-3 topical doses per day over 1-2 months, generally about 20 mL or less. For example, small volume containers include 5 mL-, 10 mL- and 15 mL-sized bottles adapted for topically administering eye drops.

Examples of surfactants according to present invention are polyethoxylated castor oils such as commercially available, and include those classified as PEG-2 to PEG-200 castor oils, as well as those classified as PEG-5 to PEG-200 hydrogenated castor oils. Such polyethoxylated castor oils include those manufactured by Rhone-Poulenc (Cranbury, N.J.) under the Alkamuls® brandand those manufactured by BASF (Parsippany, N.J.) under the Cremophor® brand. It is preferred to use the polyethoxylated castor oils classified as PEG-15 to PEG-50 castor oils, and more preferred to use PEG-30 to PEG-35 castor oils. It is most preferred to use those polyethoxylated castor oils known as Cremophor® EL and Alkamuls® EL-620; preferably Cremophor® RH-40. Examples of suitable agents that may be utilized to adjust the tonicity or osmolality of the formulations include sodium chloride, potassium chloride, mannitol, dextrose, glycerin and propylene glycol. Such agents, if present, will be employed in an amount between about 0.1 and about 10.0 wt. %.

Examples of suitable buffering agents include acetic acid, citric acid, carbonic acid, phosphoric acid, boric acid, the pharmaceutically acceptable salts of the foregoing, and tromethamine. Such buffers, if present, will be employed in an amount between about 0.001 and about 1.0 wt. %.

The compositions of the present invention may additionally include components to provide sustained release and/or comfort. Such components include high molecular weight, anionic mucomimetic polymers and gelling polysaccharides, such as those described in U.S. Pat. No. 4,861,760 (Mazuel et al), U.S. Pat. No. 4,911,920 (Jani et al.), and in commonly assigned U.S. Ser. No. 08/108,824 (Lang et al.). The contents of these patents and patent applications relating to the polymers cited above are incorporated herein by reference.

As will be appreciated by those skilled in the art, the compositions may be formulated in various dosage forms suitable for delivery of compositions. In the preferred case of topical ophthalmic delivery, the compositions may be formulated as aqueous or non-aqueous solutions, suspensions or emulsions, for example. Topically administrable ophthalmic compositions have a pH between 3.5 to 8.0 and an osmolality between 260 to 320 milliOsmoles per kilogram (mOsm/kg).

The invention will be further illustrated by the following examples, which are intended to be illustrative but not limiting.

### Example No. 1

### Preparation of Formulations:

A formulation as shown in table 1 was prepared as follows: To a clean vessel of appropriate size to which added approximately 80% of the batch volume of water. To this was sequentially added and dissolved, EDTA, Tromethamine, boric acid, mannitol, benzalkonium chloride and Cremophor® RH-40. Travoprost weighed in a glass beaker and dissolved using previously prepared solution. Next the pH of the solution was adjusted using NaOH and/or HCl, and the water was added to bring the volume to 100%. The resulting solution was then sterile filtered (0.2 μm filter).

TABLE 1

| Ingredients | Qty/ml | |
|---|---|---|
| Travoprost | 40 | mcg |
| Benzalkonium Chloride | 0.15 | mg |
| Cremophor RH-40 | 5.0 | mg |
| Mannitol | 46.0 | mg |
| Tromethamine | 1.20 | mg |
| Boric acid | 3.0 | mg |
| Disodium EDTA | 0.10 | mg |

| | |
|---|---|
| Water for injection | Adjust the final volume |
| Sodium Hydroxide | To adjust the pH |
| Hydrochloric acid | To adjust the pH |

The prepared formulations were filled in containers prepared with different resins of LDPE as shown in table 2 & 3. Either gamma sterilized or non sterilized containers were used and further studied for stability at different stability conditions. Also the formulations were filled in containers with different resins of LDPE using BFS (Blow Fill Seal) Technology and further studied for stability at different stability conditions.

TABLE 2

| | | LDPE Polymer Grade | | |
|---|---|---|---|---|
| Sl. | | Purell PE | Purell PE | Purell PE |
| No. | Parameters | 1810 E | 1840 H | 3020 D |
| 1 | Resin type | Polyethylene, | Polyethylene, | Polyethylene, |
| | | Low Density | Low Density | Low Density |
| 2 | Description | Purell PE | Purell PE | Purell PE |
| | | 1810 E is a | 1840 H is a | 3020 D is a |
| | | low density | low density | low density |
| | | polyethylene | polyethylene | polyethylene |
| | | with good | with good | with high rigidity, |
| | | felxibility and | flexibility and | good opticals and |
| | | delivered in | delivered in | good chemical |
| | | pellet form. | pellet form. | resistance. It is |
| | | | | delivered in |
| | | | | pellet form. |

TABLE 3

| | | LDPE Polymer Grade | |
|---|---|---|---|
| Sr. | | Purell PE | Purell PE |
| No. | Parameters | 3040 D | 3220 D |
| 1 | Resin type | Polyethylene, Low | Polyethylene, Low |
| | | Density | Density |
| 2 | Description | Purell PE 3040 D is a | Purell PE 3020 D is a |
| | | low-density polyethylene | low density polyethylene |
| | | with high rigidity and | with high rigidity and |
| | | good chemical resistance. | good chemical resistance. |
| | | It is delivered in pellet | It is delivered in pellet |
| | | form. | form. |

The effect of container system on stability (assay) of Travoprost Ophthalmic Solution 0.004% w/v was studied in compatibility study at different stability conditions. The results are presented in Table 4.

TABLE 4

| | | Type Resin used in LPDE containers | | | | | | | | | |
|---|---|---|---|---|---|---|---|---|---|---|---|
| | | Purell PE 1810 E ^{#}NG | | | Purell PE 1810 E *G | | | Purell PE 1840 H ^{#}NG | | | Purell PE 1840 H *G |
| Stability | | (opaque 5 ml ***TPC) | | | (opaque 5 ml ***TPC) | | | (opaque 5 ml ***TPC) | | | (opaque 5 ml ***TPC) |
| Condition | Initial | 1 W | 2 W | 4 W | 1 W | 2 W | 4 W | 1 W | 2 W | 4 W | 1 W |
| Assay- | 99.3 | 101.0 | 99.8 | 100.5 | 98.3 | 98.8 | 98.8 | 100.0 | 98.5 | 99.3 | 100.3 |
| 40° C./ | | | | | | | | | | | |
| 75% RH | | | | | | | | | | | |
| Assay- | 99.3 | 100.0 | 100.5 | 101.8 | 95.5 | 90.5 | 69.3 | 97.0 | 93.8 | 82.5 | 99.3 |
| 60° C. | | | | | | | | | | | |

| | Type Resin used in LPDE containers | | | | | | | | | | |
|---|---|---|---|---|---|---|---|---|---|---|---|
| | Purell PE 1840 H *G | | Purell PE 3020 D ^{#}NG | | | Purell PE 3020 D *G | | | Purell PE 3020 D (Trans- | | |
| Stability | (opaque 5 ml ***TPC) | | (opaque 5 ml ***TPC) | | | (opaque 5 ml) ***TPC | | | parent) (5 mL **BFS) | | |
| Condition | 2 W | 4 W | 1 W | 2 W | 4 W | 1 W | 2 W | 4 W | 1 W | 2 W | 4 W |
| Assay- | 99.3 | 99.8 | 99.5 | 99.3 | 100.0 | 99.3 | 99.5 | 99.0 | 100.3 | 99.5 | 101.0 |
| 40° C./ | | | | | | | | | | | |
| 75% RH | | | | | | | | | | | |
| Assay- | 96.0 | 84.0 | 99.8 | 99.8 | 102.8 | 97.3 | 94.3 | 86.5 | 100.0 | 101.0 | 103.8 |
| 60° C. | | | | | | | | | | | |

Note:
^{#}NG: Non gamma radiated
*G: Gamma radiated
**BFS: container prepared using Blow Fill Seal Technology
***TPC: Three piece container
W: Week

### Example No. 2

Another batch prepared with a formulation as shown in table 1 and process as described in example No. 1, these formulations were filled in containers prepared by BFS technique with LDPE Purell PE 3020 D (non gamma sterilized) containers. The effect of container system on stability (assay) of Travoprost Ophthalmic Solution 0.004% w/v was studied in compatibility study at different stability conditions over long term. The results are presented in Table No. 5.

TABLE NO. 5
Pack: BFS 5 mL Container (Purell PE 3020-D BFS container)

| | | | | | | | | | | 30° C./ | 25° C./ |
|---|---|---|---|---|---|---|---|---|---|---|---|
| Stability Condition | | 40° C./NMT 25% RH | | | | | | 60° C. | | 65% RH | 40% RH |
| Test parameter | Initial | 1 W | 2 W | 1 M | 2 M | 3 M | 6 M | 1 W | 2 W | 3 M | 3 M |
| Assay % | 101.0 | 102.6 | 103.5 | 100.3 | 101.0 | 101.0 | 99.1 | 100.9 | 103.0 | 100.5 | 99.8 |

W: Week
M: Month(s)
NMT: not more than

We claim:

1. A container for increasing the stability of prostaglandin composition comprising a prostaglandin, preservative and pharmaceutically acceptable excipients wherein the container comprises a low-density polyethylene multi-dose container.

2. The container of claim 1 wherein the prostaglandin composition comprises a prostaglandin selected from the group consisting of travoprost, latanoprost, bimatoprost, and tafluprost.

3. The container of claim 1 wherein the low density polyethylene container is a low density polyethylene bottle prepared using blow fill seal technology wherein the low density polyethylene resin is selected from the group consisting of Purell PE 1810 E, Purell PE 1840 H, Purell PE 3020 D, Purell PE 3040 D, Purell PE 3220 D.

4. The container of claim 1 wherein the prostaglandin composition is adapted for topical multi-dose ophthalmic administration.

5. The container of claim 1 wherein the low-density polyethylene multi-dose container is a small volume bottle adapted for topical ophthalmic delivery.

6. The container of claim 1 wherein the preservative is Benzalkonium Chloride.

7. The container of claim 1 wherein pharmaceutically acceptable excipients are one or more vehicles, surfactants, tonicity agents, or buffers.

8. The container of claim 1 wherein the prostaglandin composition is stable for more than six months or one year at 60° C. and 40° C./RH not more than 25%

9. The container of claim 1 wherein the wherein the prostaglandin composition is stable without refrigeration at 2-8° C.

10. The container of claim 1 wherein the wherein the prostaglandin composition is stable at room temperature up to 25° C. for more than twelve months.

11. A container capable of increasing the stability of an aqueous ophthalmic composition comprising a travoprost, latanoprost, tafluprost or bimatoprost, a preservative and pharmaceutically acceptable excipients wherein the container comprises: packaging the travoprost composition in low density polyethylene multi-dose container prepared using blow fill seal technology wherein the low density polyethylene resin is Purell PE 3020 D.

12. The container of claim 11 wherein the prostaglandin composition is stable for more than six months or one year at 60° C. and 40° C./RH not more than 25%

13. The container of claim 11 wherein the prostaglandin composition is stable without refrigeration at 2-8° C.

14. The container of claim 11 wherein the prostaglandin composition is stable at room temperature up to 25° C. for more than twelve months.

15. The container of claim 11 wherein the preservative is Benzalkonium Chloride.

16. The container of claim 11 wherein pharmaceutically acceptable excipients are one or more vehicles, surfactants, tonicity agents, or buffers.

17. A method of increasing the stability of prostaglandin composition comprising a prostaglandin, preservative and pharmaceutically acceptable excipients wherein the method comprises: packaging the prostaglandin composition in low-density polyethylene multi-dose container.

18. The method of claim 17 wherein the prostaglandin composition comprises a prostaglandin selected from the group consisting of travoprost, latanoprost, bimatoprost, and tafluprost.

19. The method of claim 17 wherein the low density polyethylene container is a low density polyethylene bottle prepared using blow fill seal technology wherein the low density polyethylene resin is selected from the group consisting of Purell PE 1810 E, Purell PE 1840 H, Purell PE 3020 D, Purell PE 3040 D, Purell PE 3220 D.

20. The method of claim 17 wherein the prostaglandin composition is adapted for topical multi-dose ophthalmic administration.

21. The method of claim 17 wherein the low-density polyethylene multi-dose container is a small volume bottle adapted for topical ophthalmic delivery.

22. The method of claim 17 wherein the preservative is Benzalkonium Chloride.

23. The method of claim 17 wherein pharmaceutically acceptable excipients are one or more vehicles, surfactants, tonicity agents, or buffers.

24. The method of claim 17 is stable for more than six months or one year at 60° C. and 40° C./RH not more than 25%

25. The method of claim 17 wherein the composition is stable without refrigeration at 2-8° C.

26. The method of claim 17 wherein the composition is stable at room temperature up to 25° C. for more than twelve months.

27. The method of claim 17 for increasing the stability of an aqueous ophthalmic composition comprising a travoprost, preservative and pharmaceutically acceptable excipients wherein the method comprises: packaging the travoprost, latanoprost, bimatoprost, or tafluprost, composition in low density polyethylene multi-dose container prepared using blow fill seal technology wherein the low density polyethylene resin is Purell PE 3020 D.

28. The method of claim 27 wherein the composition is stable for more than six months or one year at 60° C. and 40° C./RH not more than 25%

29. The method of claim 27 wherein the composition is stable without refrigeration at 2-8° C.

30. The method of claim 27 wherein the composition is stable at room temperature up to 25° C. for more than twelve months.

31. The method of claim 27 wherein the preservative is Benzalkonium Chloride.

32. The method of claim 27 wherein pharmaceutically acceptable excipients are one or more vehicles, surfactants, tonicity agents, or buffers.
